(19) United States
(12) Patent Application Publication
(10) Pub. No.: US 2009/0110610 A1
(43) Pub. Date: Apr. 30, 2009

(54) DEODORIZATION DEVICE OF COOKING APPARATUS AND COOKING APPARATUS INCLUDING DEODORIZATION DEVICE

(75) Inventors: Heung Shik CHOI, Seoul (KR); Hyun Jung KIM, Seoul (KR)

Correspondence Address: GREENBLUM & BERNSTEIN, P.L.C., 1950 ROLAND CLARKE PLACE, RESTON, VA, 20191, US

(73) Assignee: LG ELECTRONICS INC., Seoul (KR)

(21) Appl. No.: 12/182,453

(22) Filed: Jul. 30, 2008

(30) Foreign Application Priority Data
Oct. 29, 2007 (KR) 10-2007-0108960

(51) Int. Cl. A61L 9/18; A61L 9/00

(52) U.S. Cl. 422/121; 422/120

(57) ABSTRACT

A deodorization device of a cooking apparatus includes a first discharge unit disposed to intersect a flow path along which air including odor-producing materials passes, the first discharge unit having a plurality of first through holes through which the air passes; and a second discharge unit spaced apart from the first discharge unit so as to form a discharge space there between in which the air is deodorized, the second discharge unit having a plurality of second through holes through which the air from the discharge space passes.

This application claims the benefit of Korean Patent Application No. 10-2007-0108960, filed on Oct. 29, 2007, which is hereby incorporated by reference for all purposes as if fully set forth herein.

### BACKGROUND

1. Field of the Invention

The present invention relates to a deodorization device of a cooking apparatus and a cooking apparatus including the deodorization device, and more particularly, to a deodorization device of a cooking apparatus which can remove odor and odor-producing materials generated from a cooking chamber, and a cooking apparatus including the deodorization device.

2. Discussion of Related Art

When food is cooked using a conventional cooking apparatus, odor and odor-producing materials remain within the cooking apparatus even after the cooked food is removed from the cooking apparatus. Further, the odor and odor-producing materials flow out from the cooking apparatus, providing an unpleasant odor for a user.

### SUMMARY OF THE INVENTION

Accordingly, the present invention is directed towards a deodorization device of a cooking apparatus which can remove odor and odor-producing materials discharged from a cooking chamber effectively.

According to an aspect of the present invention, a deodorization device of a cooking apparatus includes a first discharge unit disposed to intersect a flow path along which air including odor-producing materials passes, the first discharge unit having a plurality of first through holes through which the air passes; and a second discharge unit spaced apart from the first discharge unit so as to form a discharge space there between in which the air is deodorized, the second discharge unit having a plurality of second through holes through which the air from the discharge space passes.

The first discharge unit may include a first discharge electrode and a first dielectric material which contacts a surface of the first discharge electrode facing toward the discharge space, and the plurality of first through holes may include first electrode through holes formed in the first discharge electrode, and first dielectric material through holes formed in the first dielectric material and connecting with the first electrode through holes. The diameter of the first electrode through holes may be larger than the diameter of the first dielectric material through holes.

The first discharge unit may include a first discharge electrode having the first through holes formed therein. The second discharge unit may include a second discharge electrode and a second dielectric material which contacts a surface of the second discharge electrode facing toward the discharge space, and the plurality of second through holes may include second dielectric material through holes formed in the second dielectric material, and second electrode through holes formed in the second discharge electrode and connecting with the second dielectric material through holes.

The second discharge unit may include a second discharge electrode and a second dielectric material which contacts a surface the second discharge electrode facing toward the discharge space, and the plurality of second through holes may be formed in portions of the second dielectric material excluding portions which contact the surface of the second discharge electrode. The second discharge unit may include a second discharge electrode spaced apart from the first discharge electrode and having the second through holes formed therein.

The plurality of first through holes may be aligned with the plurality of second through holes. The plurality of second through holes may not be aligned with the plurality of first through holes. The plurality of second through holes may be formed in portions of the second discharge unit which are offset from a portion of the first discharge unit in which the plurality of first through holes are formed.

The first discharge unit may be substantially perpendicular to the air flow path. The first discharge unit and the second discharge unit may be substantially parallel. The first discharge unit and the second discharge unit may be configured to generate plasma discharge within the discharge space for removing the odor-producing materials from the air.

The first discharge unit may include a first discharge electrode having the first through holes formed therein, and the second discharge unit may include a second discharge electrode having the second through holes formed therein. A dielectric material may be disposed between the first discharge electrode and the second discharge electrode in contact with the first discharge electrode and the second discharge electrode, and the discharge space may include dielectric material through holes formed in the dielectric material and connecting with the first through holes and the second through holes.

According to another aspect of the present invention, a deodorization device of a cooking apparatus includes a first discharge electrode disposed to intersect a flow path along which air including odor-producing materials passes, the first discharge electrode having a plurality of first electrode through holes through which the air passes; a first dielectric material contacting the first discharge electrode and having first dielectric material through holes connecting with the first electrode through holes; and a second discharge electrode spaced apart from the first dielectric material so as to form a discharge space there between in which the air is deodorized, the second discharge electrode having a plurality of second electrode through holes through which air from the discharge space passes.

One of the first discharge electrode and the second discharge electrode may be applied with a voltage, and the other may be grounded.

According to another aspect of the present invention, a deodorization device of a cooking apparatus includes a first discharge electrode disposed to intersect a flow path along which air including odor-producing materials passes, the first discharge electrode having a plurality of first electrode through holes through which the air passes; a first dielectric material contacting the first discharge electrode, and having first dielectric material through holes connecting with the first electrode through holes; a second discharge electrode spaced apart from the first dielectric material so as to form a discharge space there between in which the air is deodorized, the second discharge electrode having a plurality of second electrode through holes through which air from the discharge space passes, the plurality of second electrode through holes being disposed in alignment with the first dielectric though holes; and a second dielectric material disposed on a surface of the second discharge electrode facing toward the discharge space, and having a plurality of second dielectric material through holes through which air from the discharge space passes, the second dielectric material through holes connecting with the second electrode through holes.

According to another aspect of the present invention, a deodorization device of a cooking apparatus includes a first discharge electrode disposed to intersect a flow path along which air including odor-producing materials passes, the first discharge electrode having a plurality of first electrode through holes through which the air passes; a first dielectric material contacting the first discharge electrode, and having first dielectric material through holes connecting with the first electrode through holes; a second discharge electrode spaced apart from the first dielectric material so as to form a discharge space there between in which the air is deodorized; and a second dielectric material disposed on a surface of the second discharge electrode facing toward the discharge space and having a plurality of second dielectric material through holes through which air from the discharge space passes, wherein the plurality of second dielectric material through holes are not aligned with the first dielectric material through holes.

The plurality of second dielectric through holes may be formed in portions of the second dielectric material which are offset from a portion of the first dielectric material in which the plurality of first dielectric through holes are formed.

According to another aspect of the present invention, a deodorization device of a cooking apparatus, includes a first discharge unit disposed to intersect a flow path along which air including odor-producing materials passes, the first discharge unit having a plurality of first through holes through which the air passes; and a second discharge unit spaced apart from the first discharge unit so as to form a substantially closed discharge space there between in which the air is deodorized, the second discharge unit having a plurality of second through holes through which the air from the discharge space passes.

The plurality of second through holes may be formed in portions of the second discharge unit which are offset from a portion of the first discharge unit in which the plurality of first through holes are formed.

According to another aspect of the present invention, a cooking apparatus includes a deodorization device as set forth above disposed along a flow path; and a blower configured to force air including odor-producing materials along the flow path and through the deodorization device.

The cooking apparatus may include an ozone removal device disposed along the flow path downstream from the deodorization device for removing excess ozone from the air.

### BRIEF DESCRIPTION OF THE DRAWINGS

FIG. 1 is a sectional view of the airflow path of a cooking apparatus equipped with a deodorization device according to a first embodiment of the present invention;

FIG. 2 is a perspective view of the deodorization device, which is viewed from a direction A of FIG. 1;

FIG. 3 is a perspective view of the deodorization device, which is viewed from a direction B of FIG. 1;

FIG. 4 is a sectional view of the airflow path of a cooking apparatus equipped with a deodorization device according to a second embodiment of the present invention;

FIG. 5 is a perspective view of the deodorization device, which is viewed from a direction C of FIG. 4;

FIG. 6 is a perspective view of the deodorization device, which is viewed from a direction D of FIG. 4;

FIG. 7 is a sectional view of the airflow path of a cooking apparatus equipped with a deodorization device according to a third embodiment of the present invention;

FIG. 8 is a sectional view of the airflow path of a cooking apparatus equipped with a deodorization device according to a fourth embodiment of the present invention; and

FIG. 9 is a sectional view of the airflow path of a cooking apparatus equipped with a deodorization device according to a fifth embodiment of the present invention.

### DETAILED DESCRIPTION OF THE INVENTION

The present invention will now be described in detail in connection with specific exemplary embodiments with reference to the accompanying drawings.

The deodorization device of the present invention may be applied to any suitable type of cooking apparatus, such as a gas, electric or microwave oven. Further, the cooking apparatus of the present invention may include all kinds of devices capable of cooking food, such as a gas, electric or microwave oven.

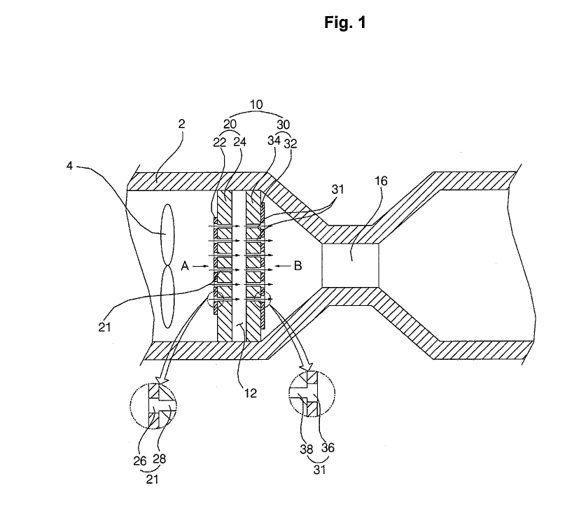

FIG. 1 is a sectional view of the airflow path of a cooking apparatus equipped with a deodorization device according to a first embodiment of the present invention.

Referring to FIG. 1, the cooking apparatus includes a cooking chamber (not shown) where food is cooked, and an airflow path 2, which connects with the cooking chamber and through which air including odor and odor-producing materials can pass. A cooling fan 4 may be disposed in the airflow path 2. The airflow path 2 may be configured to connect with the outside of the cooking apparatus.

The cooking apparatus includes a deodorization device 10 for removing odor and odor-producing materials of air that passes through the airflow path 2. The deodorization device 10 may be disposed at the rear of the cooling fan 4 along the airflow path 2. The deodorization device 10 is configured to generate ozone and ions from the air by generating plasma. The ozone and ions function to remove the odor and odor-producing materials. As shown in FIG. 1, the deodorization device 10 includes a first discharge unit 20 disposed to intersect the airflow path 2, and a second discharge unit 30 spaced apart from the first discharge unit 20. A discharge space 12 is formed between the first discharge unit 20 and the second discharge unit 30. A plurality of first through holes 21 are formed in the first discharge unit 10. The air including odor and odor-producing materials passes through the plurality of the first through holes 21. A plurality of second through holes 31 are formed in the second discharge unit 30. The air that has passed through the discharge space 12 passes through the plurality of the second through holes 31. The first discharge unit 20 may be disposed perpendicularly to the airflow path 2, and the second discharge unit 30 may be disposed in parallel to the first discharge unit 20.

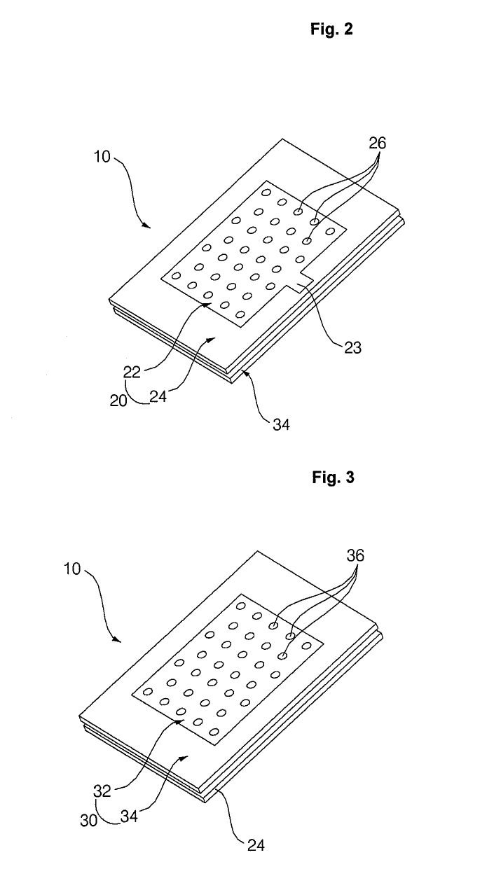

FIG. 2 is a perspective view of the deodorization device, which is viewed from a direction A of FIG. 1. FIG. 3 is a perspective view of the deodorization device, which is viewed from a direction B of FIG. 1. Referring to FIGS. 1 to 3, the first discharge unit 20 includes a first discharge electrode 22, and a first dielectric material 24. The first dielectric material 24 is disposed between the first discharge electrode 22 and the discharge space 12.

The first dielectric material 24 is coupled to an inner wall surface of the airflow path 2.

The first discharge electrode 22 may have a size smaller than that of the first dielectric material 24. The first discharge electrode 22 may be coated on a surface of the first dielectric material 24.

The plurality of first through holes 21 may include first electrode through holes 26 formed in the first discharge electrode 22, and first dielectric through holes 28 configured to connect with the first electrode through holes 26 and formed in the first dielectric material 24. The first dielectric material 24 may be formed in any suitable manner, such as from ceramics through a baking process after the first dielectric through holes 28 are perforated. The first electrode through holes 26 may be formed in any suitable manner, such as through etching. The first electrode through hole 26 may have a diameter larger than that of the first dielectric through hole 28 in order to facilitate the introduction of air blown from the cooling fan 4.

The second discharge unit 30 includes a second discharge electrode 32, and a second dielectric material 34. The second dielectric material 34 is disposed between the second discharge electrode 32 and the discharge space 12.

The second dielectric material 34 is coupled to an inner wall surface of the airflow path 2. The second dielectric material 34 may be spaced apart from the first dielectric material 24 at a predetermined interval. The first and second dielectric materials 24 and 34 may have the same thickness. A distance between the first dielectric material 24 and the second dielectric material 34 may be identical to or similar to the thickness of the first and second dielectric materials 24 and 34.

The second discharge electrode 32 may have a size smaller than that of the second dielectric material 34. The second discharge electrode 32 may be coated on a surface of the second dielectric material 34. The first discharge electrode 22 and the second discharge electrode 32 may have the same size, or one of the first discharge electrode 22 and the second discharge electrode 32 may have a size larger than that of the other of the first discharge electrode 22 and the second discharge electrode 32 in consideration of stability.

The first discharge electrode 22 is applied with a voltage from a voltage application unit (not shown), and the second discharge electrode 32 is grounded. As shown in FIG. 2, a connection unit 23, which is electrically connected to the voltage application unit in any suitable manner, such as through an electric wire, is projected from the first discharge electrode 22.

The plurality of second through holes 31 includes second dielectric through holes 38 formed in the second dielectric material 34, and second electrode through holes 36 configured to connect with the second dielectric through holes 38 and formed in the second discharge electrode 32. The second dielectric material 34 may be formed in any suitable manner, such as from ceramics through a baking process after the second dielectric through holes 38 are perforated. The second electrode through holes 36 may be formed in any suitable manner, such as through etching. The second electrode through hole 36 may have a diameter larger than that of the second dielectric through hole 38 in order to facilitate the flow of air discharged from the discharge space 12.

As shown in FIG. 1, an ozone removal device 16 may be disposed at the rear of the deodorization device 10 along the airflow path 12. The ozone removal device 16 is a device for removing ozone remaining in the air after it has flowed through the discharge space 12. The ozone removal device 16 functions to break down ozone remaining in the air from the deodorization device 10 by heating air to a predetermined temperature or higher. However, the present invention is not limited to the above construction. For example, the ozone removal device 16 may remove ozone in any suitable manner.

An operation of the deodorization device constructed above according to a first embodiment of the present invention is described below.

When food is cooked in the cooking chamber, odor and odor-producing materials are generated. In order to ventilate the cooking chamber, the cooling fan 4 is operated. The cooling fan 4 enables air, including the odor and odor-producing materials within the cooking chamber, to pass through the airflow path 2. The air sent from the cooling fan 4 is introduced to the discharge space 12 through the first electrode through holes 26 and the first dielectric through holes 28.

When the deodorization device 10 is actuated, a voltage is applied to the first discharge electrode 22, so that plasma discharge is generated within the discharge space 12, between the first discharge electrode 22 and the second discharge electrode 32. Upon plasma discharge, ozone and ions are generated from the air within the discharge space 12. Various kinds of ions, including negative ions such as hydroxide (OH−) ions, may be generated. The ions and ozone react with the odor and odor-producing materials introduced to the discharge space 12, and decompose and remove the odor and odor-producing materials. Since the discharge space 12 is limited by the first discharge unit 20 and the second discharge unit 30, all air including odor and odor-producing material passes through the discharge space 12. Accordingly, odor and odor-producing materials in all of the air which passes through the airflow path 2 can be removed within the discharge space 12. The air from which the odor and odor-producing materials have been removed within the discharge space 12 is discharged from the discharge space 12 through the second dielectric through holes 38 and the second electrode through holes 36.

A main source material of the smell is a mixture of C—H, wherein the ions and ozone disconnect C—H bond in the source material. The amount of removed smell is determined depending on the amount of generated ions and ozone, the reaction time of the smell, and the like.

Furthermore, the moisture is condensed and small aerosol particles are produced during the plasma discharge, and the condensed moisture and produced small aerosol particles play a role to remove the smell. Especially, the small aerosol particles directly react with the material that generates the smell thereby to produce a large quantity of CH₃—S radicals. The CH₃—S radicals disconnect the C—H bond, thus removing the smell.

Ozone may remain in the air from which the odor and odor-producing materials have been removed. The air passing through the deodorization device 10 passes through the ozone removal device 16. The air from which ozone has been removed by the ozone removal device 16 is discharged to the outside through the airflow path 2.

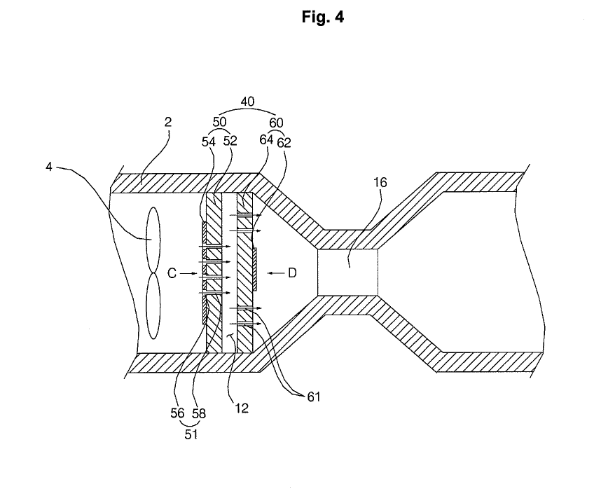

FIG. 4 is a sectional view of the airflow path of a cooking apparatus equipped with a deodorization device according to a second embodiment of the present invention

Referring to FIG. 4, a deodorization device 40 according to a second embodiment of the present invention includes a first discharge unit 50 disposed to intersect an airflow path and having a plurality of first through holes 51 through which air including odor and odor-producing materials pass, and a second discharge unit 60 spaced apart from the first discharge unit 50 and forming a discharge space 12 along with the first discharge unit 50 therebetween. A plurality of second through holes 61 through which air, which has passed through the discharge space 12, passes are formed in the second discharge unit 60. The deodorization device 40 according to the second embodiment of the present invention may have the same or similar construction and operation as those of the deodorization device according to the first embodiment of the present invention except that the second through holes 61 are offset in a diagonal direction with respect to the first through holes 51. Redundant description with the first embodiment of the present invention will be omitted.

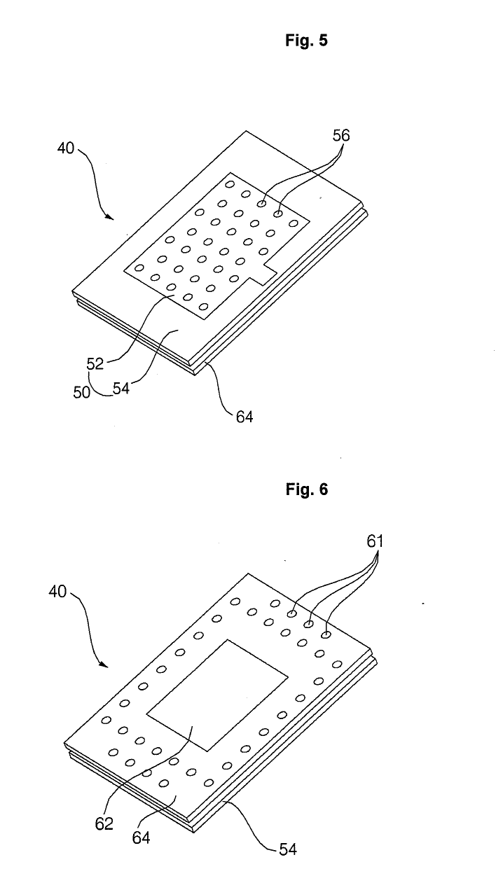

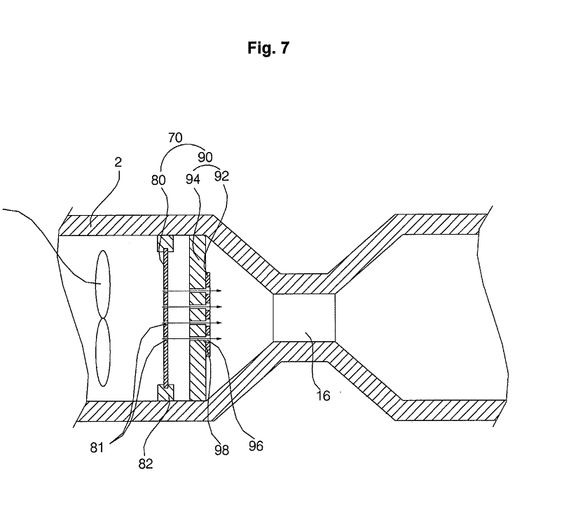

FIG. 5 is a perspective view of the deodorization device, which is viewed from a direction C of FIG. 4. FIG. 6 is a perspective view of the deodorization device, which is viewed from a direction D of FIG. 4.

Referring to FIGS. 4 to 6, the first discharge unit 50 includes a first discharge electrode 52, and a first dielectric material 54 coming in contact with the first discharge electrode 52. The plurality of first through holes 51 include first electrode through holes 56 formed in the first discharge electrode 52, and first dielectric through holes 58 formed in the first dielectric material 54.

The second discharge unit 60 includes a second discharge electrode 62, and a second dielectric material 64 coming in contact with the second discharge electrode 62. The plurality of second through holes 61 may be formed only in the second dielectric material 64. In other words, as shown in FIG. 6, the second through holes 61 may be formed in the remaining portions excluding the portions where the second dielectric material 64 comes in contact with the second discharge electrode 62. The second through holes 61 are formed in the second dielectric material 64 in a diagonal direction with respect to the first through holes 51. In other words, the second through holes 61 are offset from, and not aligned with, the first through holes 51.

However, the present invention is not limited to the above construction. For example, the second through holes 61 are offset, or disposed in a diagonal direction, with respect to the first through holes 51, but may be formed in the second discharge electrode 62 and the second dielectric material 64, respectively.

The deodorization device 40 constructed as described above according to the second embodiment of the present invention has the first through holes 51 and the second through holes 61 in portions of the first and second discharge unit 50, 60 which are offset and not overlapping. In this manner the air remains in the discharge space 12 for a longer period of time, and more of the ozone remains in the discharge space 12. Thus, since the duration of time during which air comes in contact with negative ions or ozone within the discharge space 12 is lengthened, odor and odor-producing materials can be removed more effectively.

FIG. 7 is a sectional view of the airflow path of a cooking apparatus equipped with a deodorization device according to a third embodiment of the present invention.

Referring to FIG. 7, a deodorization device 70 according to a third embodiment of the present invention includes a first discharge unit 80 disposed along an airflow path 2, and a second discharge unit 90 spaced apart from the first discharge unit 80. The deodorization device 70 according to a third embodiment of the present invention may have the same or similar construction and operation as those of the deodorization device according to the first embodiment of the present invention except that one of the first discharge unit 80 and the second discharge unit 90 includes only a discharge electrode and the other includes a discharge electrode and a dielectric material. Redundant description with the first embodiment of the present invention will be omitted.

The first discharge unit 80 is provided as a first discharge electrode 80 having first through holes 81 formed therein. A support 82 for supporting the first discharge electrode 80 is disposed in the airflow path 2.

The second discharge unit 90 includes a second discharge electrode 92, and a second dielectric material 94 in contact with the second discharge electrode 92. Second electrode through holes 96 are formed in the second discharge electrode 92, and second dielectric through holes 98 are formed in the second dielectric material 94.

The first through holes 81 and the second dielectric through holes 98 may be disposed in alignment with each other. However, the present invention is not limited to the above construction. For example, the second dielectric through holes 98 may be disposed offset in a diagonal direction with respect to the first through holes 81.

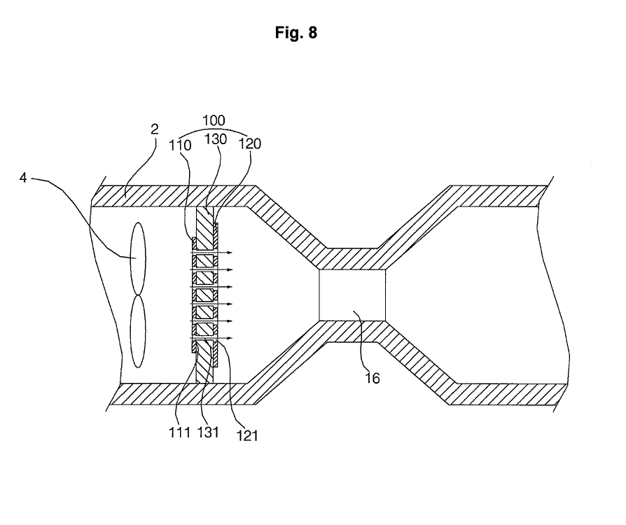

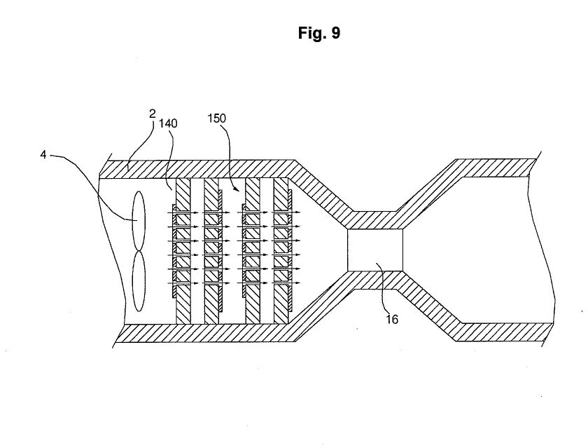

FIG. 8 is a sectional view of the airflow path of a cooking apparatus equipped with a deodorization device according to a fourth embodiment of the present invention.

Referring to FIG. 8, a deodorization device 100 according to a fourth embodiment of the present invention may have the same or similar construction and operation as those of the deodorization device according to the first embodiment of the present invention except that it includes a first discharge electrode 110 having first through holes 111 formed therein, a second discharge electrode 120 having second through holes 121 formed therein, and a dielectric material 130 formed between the first discharge electrode 110 and the second discharge electrode 120 in such a way as to contact with them. Redundant description with the first embodiment of the present invention will be omitted.

The dielectric material 130 has one side in contact with the first discharge electrode 110 and the other side in contact with the second discharge electrode 120. Dielectric through holes 131 whic connect with the first through holes 111 and the second through holes 121 are formed in the dielectric material 130. The dielectric through holes 131 serve as a discharge space.

Thus, if a voltage is applied to the first discharge electrode 110, plasma discharge is generated within the dielectric through holes 131. As air including odor and odor-producing materials passes through the dielectric through holes 131, the odor and odor-producing materials included in the air can be removed through reaction with negative ions and ozone generated by the plasma discharge.

FIG. 9 is a sectional view of the airflow path of a cooking apparatus equipped with a deodorization device according to a fifth embodiment of the present invention.

Referring to FIG. 9, a deodorization device according to a fifth embodiment of the present invention may have the same or similar construction and operation as those of the deodorization device according to the first embodiment of the present invention except that a plurality of deodorization devices 140 and 150 are disposed in series along the airflow path. Redundant description with the first embodiment of the present invention will be omitted.

The deodorization device includes the first deodorization device 140 disposed at the rear of a cooling fan 4, and the second deodorization device 150 disposed at the rear of the first deodorization device 140. Thus, odor and odor-producing materials, included in air sent from the cooling fan 4, are primarily filtered while passing through the first deodorization device 140. The air that has passed through the first deodorization device 140 is introduced to the second deodorization device 150, so that the remaining odor and odor-producing materials can be filtered secondarily.

Although each embodiment has been depicted as including an ozone removal device 16, the ozone removal device is not necessarily required, and may be omitted. In the deodorization device of the cooking apparatus and the cooking apparatus including the deodorization device according to the present invention, the discharge space is limited between the first discharge unit and the second discharge unit, and air including odor and odor-producing materials passes through the discharge space. Accordingly, the odor and odor-producing materials can be removed within the limited discharge space more effectively. Further, since plasma discharge is generated only within the limited discharge space, diffusion of ozone generated upon plasma discharge can be reduced.

While the invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

The foregoing embodiments and advantages are merely exemplary and are not to be construed as limiting the present invention. The present teaching can be readily applied to other types of apparatuses. The description of the present invention is intended to be illustrative, and not to limit the scope of the claims. Many alternatives, modifications, and variations will be apparent to those skilled in the art.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The above disclosed subject matter is to be considered illustrative, and not restrictive, and the appended claims are intended to cover all such modifications, enhancements, and other embodiments which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

Although the invention has been described with reference to several exemplary embodiments, it is understood that the words that have been used are words of description and illustration, rather than words of limitation. As the present invention may be embodied in several forms without departing from the spirit or essential characteristics thereof, it should also be understood that the above-described embodiments are not limited by any of the details of the foregoing description, unless otherwise specified. Rather, the above-described embodiments should be construed broadly within the spirit and scope of the present invention as defined in the appended claims. Therefore, changes may be made within the metes and bounds of the appended claims, as presently stated and as amended, without departing from the scope and spirit of the invention in its aspects.

What is claimed is:

1. A deodorization device of a cooking apparatus, comprising:
a first discharge unit disposed to intersect a flow path along which air including odor-producing materials passes, the first discharge unit having a plurality of first through holes through which the air passes; and
a second discharge unit spaced apart from the first discharge unit so as to form a discharge space there between in which the air is deodorized, the second discharge unit having a plurality of second through holes through which the air from the discharge space passes.

2. The deodorization device of claim 1, wherein:
the first discharge unit comprises a first discharge electrode and a first dielectric material which contacts a surface of the first discharge electrode facing toward the discharge space, and
the plurality of first through holes comprise first electrode through holes formed in the first discharge electrode, and first dielectric material through holes formed in the first dielectric material and connecting with the first electrode through holes.

3. The deodorization device of claim 2, wherein the diameter of the first electrode through holes are larger than the diameter of the first dielectric material through holes.

4. The deodorization device of claim 1, wherein the first discharge unit comprises a first discharge electrode having the first through holes formed therein.

5. The deodorization device of claim 1, wherein:
the second discharge unit comprises a second discharge electrode and a second dielectric material which contacts a surface of the second discharge electrode facing toward the discharge space, and
the plurality of second through holes comprise second dielectric material through holes formed in the second dielectric material, and second electrode through holes formed in the second discharge electrode and connecting with the second dielectric material through holes.

6. The deodorization device of claim 1, wherein:
the second discharge unit comprises a second discharge electrode and a second dielectric material which contacts a surface the second discharge electrode facing toward the discharge space, and
the plurality of second through holes are formed in portions of the second dielectric material excluding portions which contact the surface of the second discharge electrode.

7. The deodorization device of claim 2, wherein the second discharge unit comprises a second discharge electrode spaced apart from the first discharge electrode and having the second through holes formed therein.

8. The deodorization device of claim 1, wherein the plurality of first through holes are aligned with the plurality of second through holes.

9. The deodorization device of claim 1, wherein the plurality of second through holes are not aligned with the plurality of first through holes.

10. The deodorization device of claim 1, wherein the plurality of second through holes are formed in portions of the second discharge unit which are offset from a portion of the first discharge unit in which the plurality of first through holes are formed.

11. The deodorization device of claim 1, wherein the first discharge unit is substantially perpendicular to the air flow path.

12. The deodorization device of claim 1, wherein the first discharge unit and the second discharge unit are substantially parallel.

13. The deodorization device of claim 1, wherein the first discharge unit and the second discharge unit are configured to generate plasma discharge within the discharge space for removing the odor-producing materials from the air.

14. The deodorization device of claim 1, wherein:
the first discharge unit comprises a first discharge electrode having the first through holes formed therein, and the second discharge unit comprises a second discharge electrode having the second through holes formed therein,
a dielectric material is disposed between the first discharge electrode and the second discharge electrode in contact with the first discharge electrode and the second discharge electrode, and
the discharge space comprises dielectric material through holes formed in the dielectric material and connecting with the first through holes and the second through holes.

15. A deodorization device of a cooking apparatus, comprising:
a first discharge electrode disposed to intersect a flow path along which air including odor-producing materials passes, the first discharge electrode having a plurality of first electrode through holes through which the air passes;
a first dielectric material contacting the first discharge electrode and having first dielectric material through holes connecting with the first electrode through holes; and
a second discharge electrode spaced apart from the first dielectric material so as to form a discharge space there between in which the air is deodorized, the second discharge electrode having a plurality of second electrode through holes through which air from the discharge space passes.

16. The deodorization device of claim 15, wherein one of the first discharge electrode and the second discharge electrode is applied with a voltage, and the other is grounded.

17. A deodorization device of a cooking apparatus, comprising:
a first discharge electrode disposed to intersect a flow path along which air including odor-producing materials passes, the first discharge electrode having a plurality of first electrode through holes through which the air passes;
a first dielectric material contacting the first discharge electrode, and having first dielectric material through holes connecting with the first electrode through holes;
a second discharge electrode spaced apart from the first dielectric material so as to form a discharge space there between in which the air is deodorized, the second discharge electrode having a plurality of second electrode through holes through which air from the discharge space passes, the plurality of second electrode through holes being disposed in alignment with the first dielectric though holes; and
a second dielectric material disposed on a surface of the second discharge electrode facing toward the discharge space, and having a plurality of second dielectric material through holes through which air from the discharge space passes, the second dielectric material through holes connecting with the second electrode through holes.

18. A deodorization device of a cooking apparatus, comprising:
a first discharge electrode disposed to intersect a flow path along which air including odor-producing materials passes, the first discharge electrode having a plurality of first electrode through holes through which the air passes;
a first dielectric material contacting the first discharge electrode, and having first dielectric material through holes connecting with the first electrode through holes;
a second discharge electrode spaced apart from the first dielectric material so as to form a discharge space there between in which the air is deodorized; and
a second dielectric material disposed on a surface of the second discharge electrode facing toward the discharge space and having a plurality of second dielectric material through holes through which air from the discharge space passes, wherein the plurality of second dielectric material through holes are not aligned with the first dielectric material through holes.

19. The deodorization device of claim 18, wherein the plurality of second dielectric through holes are formed in portions of the second dielectric material which are offset from a portion of the first dielectric material in which the plurality of first dielectric through holes are formed.

20. A deodorization device of a cooking apparatus, comprising:
a first discharge unit disposed to intersect a flow path along which air including odor-producing materials passes, the first discharge unit having a plurality of first through holes through which the air passes; and
a second discharge unit spaced apart from the first discharge unit so as to form a substantially closed discharge space there between in which the air is deodorized, the second discharge unit having a plurality of second through holes through which the air from the discharge space passes.

21. The deodorization device of claim 20, wherein the plurality of second through holes are formed in portions of the second discharge unit which are offset from a portion of the first discharge unit in which the plurality of first through holes are formed.

22. A cooking apparatus comprising:
a deodorization device according to claim 1 disposed along a flow path; and
a blower configured to force air including odor-producing materials along the flow path and through the deodorization device.

23. The cooking apparatus of claim 22, further comprising an ozone removal device disposed along the flow path downstream from the deodorization device for removing excess ozone from the air.
